(19) United States
(12) Patent Application Publication
(10) Pub. No.: US 2004/0137426 A1
(43) Pub. Date: Jul. 15, 2004

(54) Gp41 peptides and methods based thereon for inhibiting HIV fusion to target cells

(76) Inventors: Pierre-Francois Serres, Saint Genis Leval (FR); Joseph D. Mosca, Ellicott City, MD (US)

Correspondence Address: OLIFF & BERRIDGE, PLC, P.O. BOX 19928, ALEXANDRIA, VA, 22320, US

(21) Appl. No.: 10/457,780

(22) Filed: Jun. 10, 2003

Related U.S. Application Data
(60) Provisional application No. 60/446,268, filed on Feb. 11, 2003.
(60) Provisional application No. 60/413,919, filed on Sep. 27, 2002.
(60) Provisional application No. 60/386,754, filed on Jun. 10, 2002.

(51) Int. Cl. C12Q 1/70; A61K 38/16; C07K 14/16

(52) U.S. Cl. 435/5; 514/12; 530/324

(57) ABSTRACT

The present invention provides peptides which are based on the HIV GP41 protein and have inhibitory activity against the fusion of HIV virus to its target cells. The invention also provides therapeutic methods based on the administration of the peptides of the invention to a subject infected with the HIV virus.

### RELATED APPLICATIONS

The present application claims priority to U.S. Provisional Application Serial No. 60/386,754 filed Jun. 10, 2002; U.S. Provisional Application Serial No. 60/413,919 filed Sep. 27, 2002 and U.S. Provisional Application Serial No. 60/446,268 filed Feb. 11, 2003. The contents of the three applications are incorporated by reference in their entirety.

### FIELD OF THE INVENTION

The present invention relates the identification of peptides that inhibit the fusion of the HIV virus to a target cell thereby providing new therapies against HIV infection.

### DESCRIPTION OF RELATED ART

Human immunodeficiency viruses (HIV) of type 1 and 2 are lentiviruses from the family of retroviruses that are believed to cause acquired immunodeficiency syndrome (AIDS). Human T lymphotropic viruses (HTLV) of type 1 and 2 are also human retroviruses and cause adult T cell leukemia, neurodegenerative diseases, and immunodeficiency. Thus, there are several types a of pathogenic human retroviruses and as used herein HIV refers to them generically. The transmission of HIV through sexual contact and during pregnancy accounts for up to 90% of AIDS cases worldwide. This transmission is initiated by the passage of HIV across the mucosal barrier of sexual organs or placenta when exposed to infectious body fluids such as semen, vaginal secretions or blood. The remaining AIDS cases are due to the transfusion of HIV-contaminated blood, needle sharing among intravenous drug users, accidental exposure to HIV-contaminated body fluids during invasive procedures, and other situations wherein infectious virus can come into direct contact with susceptible human tissues.

HIV-1 damages the immune system by infecting and depleting T helper/inducer lymphocytes (hereinafter referred to as “T cells”). T cells are essential because they control the production of antibodies by the B cells, the maturation of cytotoxic T lymphocytes (killer T cells), the maturation and activity of macrophages and natural killer cells, and directly and indirectly, numerous other regulator and effector functions of the immune system.

Infection of a T cell occurs through interaction between an epitope borne by HIV-1 and a receptor site which is located on the T cell surface. This receptor site on the T cell is a protein molecule known as the CD4 antigen. The epitope on HIV-1 is borne by the external envelope glycoprotein gp120 (molecular weight about 120,000 daltons).

The glycoprotein gp120 is produced when a precursor glycoprotein gp 160, made in the HIV-1-infected T cell, is cleaved apart into a transmembrane portion gp41 (molecular weight about 41,000 daltons) and gp120. Glycoprotein gp41 spans through the membrane lipid bilayer of the virions and of the infected cells and its exterior portion is associated with gp120 through noncovalent binding. Glycoprotein gp120 bears a site which fuses with target cells, whereby the genetic material of the virus enters the cell.

Effective compounds with anti-HIV activity that could be used to prevent and/or treat AIDS are still lacking. Although initially the results with certain anti-HIV agents, e.g., azidothymidine (AZT) appeared to be promising, it has become clear that toxicity or undesirable side effects of such agents are incompatible with their antiviral activity when used at an effective pharmaceutical concentration (Bourinbaiar & Fruhstorfer, Cell Pharmacol AIDS Sci, 3:163-9, 1996). Similar concerns regarding toxicity have arisen upon use of recently introduced new drugs, such as HIV protease inhibitors. Thus, present methods of preventing and treating AIDS and HIV infection are limited and it is thus obvious that better alternative compounds devoid of toxicity and of undesirable side effects must be sought.

A classical approach to inhibiting viral replication is to block the assembly of the viral components into virus particles by providing an excess of a mutant form of the normal viral proteins, the mutant protein is referred to as a trans-dominant inhibitor. It is generally thought that the mutant protein interferes with critical protein to protein interactions that are required to build up the structure of the viral particle.

However other mechanisms can be envisaged such as the interference with cellular factors, such as the cyclophilins (Luban et al 1993) or protein kinases (Yu et al 1995) that might be required for proper virus particle formation. The approach was first proposed as a strategy for blocking HIV replication by Baltimore (1988) who subsequently demonstrated that the expression of a truncated form of one of the Gag proteins, p24, reduced viral replication (Trono et al 1989). This general approach has been developed by others (e.g. Lee and Linial 1995; Neidrig et al 1995; Lori et al 1994; Smythe et al 1994; Karacostas et al 1993) and is reviewed by Modrow et al 1994 & Meile and Lever (1996). The approach is however not always successful. For example Miele and Lever (1996) failed to protect cells against infection by using a modified p55. In addition the expression of full length proteins may be toxic as observed for p55 (Miele and Modrow op. cit.) or have other effects such as inhibition of lymphocyte proliferation as observed for wild type p¹⁷ (Hofnan et al 1994). In addition in some cases the inhibition of viral replication by trans-dominant mutants of Gag was not dramatic. For example a truncated form of p24 was poorly effective in one study (Lori et al 1994). There is therefore a need for more effective inhibitors of viral assembly. There is also a need for inhibitors which are non-toxic or have acceptably low toxicity.

High plasma levels of human immunodeficiency virus type 1 (HIV-1) RNA are found during primary infection with HIV-1, the seroconversion illness, [C. Baumberger et al, AIDS, 7:(suppl 2):S59 (1993); M. S. Saag et al, Nature Med., 2:625 (1996)], after which they subside as the immune response controls the infection to a variable extent. Post seroconversion, lower but detectable levels of plasma HIV-1 RNA are present, and these levels rise with disease progression to again attain high levels at the AIDS stage [M. S. Saag et al, Nature Med., 2:265 (1996)]. Approximately 50% of subjects have a symptomatic illness at seroconversion [B. Tindall and D. A. Cooper, AIDS, 5:1 (1991)] and symptomatic seroconversion is associated with an increased risk for the development of AIDS, probably because a severe primary illness is likely related to an early and extensive spread of HIV.

Inhibition of viral multiplication during the initial infection will likely reduce the subsequent development of chronic viremia leading to AIDS. Current medical practice, with administration of antiviral drugs for defined “at risk” situations, such as needle sticks with contaminated blood or pregnancy in HIV infected mothers, supports this concept.

Post seroconversion levels of HIV-1 RNA in plasma have proven to be the most powerful prognosticator of the likelihood of progression to AIDS [J. W. Mellors et al, Science, 272:1167 (1996); M. S. Saag et al, Nature Med., 2:265 (1996); R. W. Coombs et al, J. Lnf. Dis, 174:704 (1996); S. L. Welles et al, J. Inf. Dis., 174:696 (1990)]. Other measures of viral load, such as cellular RNA [K. Saksela et al, Proc. Natl. Acad. Sci. USA, 91:1104 (1994)] and cellular HUV proviral DNA [T-H. Lee et al, J. Acq. 1 mm. Def. Syndromes, 7:381 (1994)] similarly establish the importance of the initial infection in establishing viral loads that determine future disease progression.

Thus, any intervention that inhibits HIV-1 infectivity during initial infection and/or lowers viral load post sero-conversion is likely to have a favorable influence on the eventual outcome, delaying or preventing progression to AIDS.

A variety of methods are now employed to treat patients infected with human immunodeficiency virus (HIV-1), including treatment with certain combinations of protease inhibitor drugs. Unfortunately, however, this type of treatment is associated with serious side effects in some patients. Alternatively, vaccines are under development for control of the spread of HIV-1 to uninfected humans. Other approaches to HIV-1 treatment have focused on the transactivating (tat) gene of HIV-1, which produces a protein (Tat) essential for transcription of the virus. The tat gene and its protein have been sequenced and examined for involvement in proposed treatments of HIV [see, e.g., U.S. Pat. No. 5,158,877; U.S. Pat. No. 5,238,882; U.S. Pat. No. 5,110,802; International Patent Application No. WO92/07871, published May 14, 1992; International Patent Application No. WO91/10453, published Jul. 25, 1991; International Patent Application No. WO91/09958, published Jul. 11, 1991; International Patent Application No. WO87/02989, published May 21, 1987]. Tat protein is released extracellularly, making it available to be taken up by other infected cells to enhance transcription of HIV-1 in the cells and to be taken up by noninfected cells, altering host cell gene activations and rendering the cells susceptible to infection by the virus. Uptake of Tat by cells is very strong, and has been reported as mediated by a short basic sequence of the protein [S. Fawell et al., Proc. Natl. Acad. Sci., USA, 91:664-668 (1994)].

Despite the growing knowledge about HIV-1 disease progression, there remains a need in the art for the development of compositions and methods for treatment of HIV-1, both prophylactically and therapeutically, which are useful to lower the viral levels of HIV-1 for the treatment and possible prevention of the subsequent, generally fatal, AIDS disease.

Fusion of the HIV envelope with the target cell membrane is a critical step of HIV entry into the target cell. Fusion inhibitors block the first step in virus infection of human cells and represent one of the most exciting new areas for antiviral drug development. The HIV envelope glycoprotein gp41 plays an important role in the fusion of viral and target cell membranes and serves as an attractive target for development of HIV fusion inhibitors. The extracellular domain of gp41 contains three important functional regions, i.e. fusion peptide (FP), N- and C-terminal heptad repeats (NHR and CHR, respectively). The FP region is composed of hydrophobic, glycine-rich residues that are essential for the initial penetration of the target cell membrane. NHR and CHR regions consist of hydrophobic residues, which have the tendency to form a-helical coiled coils. One proposed scenario for the mechanism of fusion of the virus to a target cell is based on the proposition that during the process of fusion of HIV or HIV-infected cells with uninfected cells, FP inserts into the target cell membrane and subsequently the NHR and CHR regions change conformations and associate with each other to form a fusion-active gp41 core. Peptides derived from NHR and CHR regions, designated N and C-peptides, respectively, have potent inhibitory activity against HIV fusion by binding to the CHR and NHR regions, respectively, to prevent the formation of the fusion-active gp41 core. C-peptides may also bind to FP, thereby blocking its insertion into the target cell membrane. A number of synthetic peptides including T-20 corresponding to specific regions within the carboxyl-terminal heptad repeat region (HR2) of the human immunodeficiency virus type 1 (HIV-1) transmembrane protein (TM) gp41, demonstrate potent inhibition of viral replication of HIV-1 both in vitro and in vivo. T-20 (Trimeris, Roche) a C-peptide, has shown potent in vivo activity against HIV infection and has been approved by the FDA as the first peptide HIV fusion inhibitory drug. However, this HIV fusion inhibitor peptide may have limitations associated with possible viral resistance.

Therefore, there remains a need for new fusion inhibiting peptides and anti-HIV therapeutic methods based thereon which provide an alternative to the present HIV fusion inhibitor peptides.

### SUMMARY AND OBJECTS OF THE INVENTION

The present invention is based on Gp41 peptides that are rationally designed to provide enhanced inhibition of virus fusion to target cells. The peptides are designed based on one or more criteria relating to the level of conservation of particular regions of the Gp41 molecule, fine tuning of the degree of hydrophobicity, hydrophilicity, degree of immunogenicity of the peptides, the portion of the peptides that is exposed to solvent, proportion of helix forming amino acids (helical content or propensity of the peptide to form a helix), and three dimensional relationship to related host molecules such as IL-2 (e.g., IL-2/Gp41 mimicry). The peptides are proposed to bind specific portions of the amino-terminal region, preventing membrane fusion and ultimately HIV infection of the target cells. One consideration in the design and use of the new peptides is a molecular mimicry between the trimeric ectodomain of the gp41 protein of HIV-1 and IL-2. In a particular aspect of the invention, peptides are designed based on the IL-2 sequence according to criteria that involve the structural similarities between Gp41 and IL-2.

In one aspect, the subject invention provides novel peptides having high activity in blocking the fusion of the HIV virus to target cells. The peptides are rationally designed to reduce or totally eliminate the deleterious effects associated with virus mutation and drug resistance. The peptides are designed taking into account conservation criteria, as well as a number of physical and structural properties.

The present invention provides novel peptides that exhibit high inhibitory activity against the fusion of HIV virus to its target cell and therefore high inhibitory activity of HIV infection. In particular, peptides comprising the sequence of the tryptophan-rich domain WXXXXWXWX, where X is any amino acid

As well, the invention provides novel peptides corresponding to SEQ ID NOs 3-50 and 55-72 which have shown improved HIV infection inhibitory activity.

The invention also provides therapeutic methods and methods of inhibiting virus fusion to a target cell based on the peptides of the invention, particularly, peptides having a sequence selected from SEQ ID Nos 3-50 and 55-72.

The present invention provides a polypeptide having a sequence selected from the group consisting of:

| | |
|---|---|
| SLEQIWNNMTWMEWEREIDNYTSLIYSLI (S29I) | SEQ ID NO 3 |
| ILSYILSTYNDIEREWEMWTMNNWIQELS (r-S29I) | SEQ ID NO 4 |
| SLEQIWNNMT Nal2 MEWEREIDNYTSLIYSLI S29I(A) | SEQ ID NO 5 |
| ILSYILSTYNDIEREWEMNal2TMNNWIQELS (r-S29I(A)) | SEQ ID NO 6 |
| Ac-SLEQIWNNMTWME Nal2 EREIDNYTSLIYSLI (S29I(B)) | SEQ ID NO 7 |
| ILSYILSTYNDIEREWEMWT Nle NNWIQELS (r-S29I(B)) | SEQ ID NO 8 |
| SLEQIWNN Nle TWMEWEREIDNYTSLIYSLI (S29I(C)) | SEQ ID NO 9 |
| ILSYILSTYNDIEREWEMWT Nle NNWIQELS (r-S29I(C)) | SEQ ID NO 10 |
| Ac-SLEQI Nal2 NNMTWMEWEREIDNYTSLIYSLI (S29I(D)) | SEQ ID NO 11 |
| ILSYILSTYNDIEREWEMWTMNN Nal2 IQELS (r-S29I(D)) | SEQ ID NO 12 |
| SLEQIWNNMTW Nle EWEREIDNYTSLIYSLI (S29I(E)) | SEQ ID NO 13 |
| ILSYILSTYNDIEREWEMWTMNNWIQELS (r-S29I(E)) | SEQ ID NO 14 |
| NNMTWMEWEREIDNYTSLIYSLIEESQNQQEKNEQE (N36E) | SEQ ID NO 15 |
| EQENKEQQNQSEEILSYILSTYNDIEREWEMWTMNN (r-N36E) | SEQ ID NO 16 |
| NNMTWQEWEQKITAYTSLIYSLLEQAQIQQEKNEYE (N36E(ch. 10)) | SEQ ID NO 17 |
| EYENKEQQIQAQELLSYILSTYATIKQEWEQWTMNN (r-N36E(CH. 10)) | SEQ ID NO 18 |
| NNMTWQEWEQKITAYTSLIHSLLEQAQIQQEKNEYE (N36E(ch. 11)) | SEQ ID NO 19 |
| EYENKEQQIQAQELLSHILSTYATIKQEWEQWTMNN (r-N36E(CH. 11)) | SEQ ID NO 20 |
| NNMTWMEWEREIDNYTSLIHSLIEESQNQQEKNEQE (N36E(ch. 1)) | SEQ ID NO 21 |
| EQENKEQQNQSEEILSHILSTYNDIEREWEMWTMNN (r-N36E(ch. 1)) | SEQ ID NO 22 |
| NNMTWQEWEQKITALLEQAQIQQEKNEYE (N36E(del. 7)) | SEQ ID NO 23 |
| EYENKEQQIQAQELLATIKQEWEQWTMNN (r-N36E(del. 7)) | SEQ ID NO 24 |
| NNMTWQEWEQKITAYTSLIHSLIEESQNQQEKNEQE (N36E(ch. 6)) | SEQ ID NO 25 |
| EQENKEQQNQSEEILSHILSTYATIKQEWEQWTMNN (r-N36E(ch. 6)) | SEQ ID NO 26 |
| WSNKSLEQIWNNMTWMEWEREIDNYTSLIYSLIEESQ (W37Q) | SEQ ID NO 27 |
| QSEEILSYILSTYNDIEREWEMWTMNNWIQELSKNSW (r-W37Q) | SEQ ID NO 28 |
| WSNKSLEQIWNNMTWMEWEREIDNYTSLIHSLIEESQ (W37Q(ch. 1)) | SEQ ID NO 29 |
| QSEEILSHILSTYNDIEREWEMWTMNNWIQELSKNSW (r-W37Q(ch. 1)) | SEQ ID NO 30 |
| WSNKSLEQIWNNMTWQEWEQKITAYTSLIYSLLEQAQ (W37Q(ch. 8)) | SEQ ID NO 31 |
| QAQELLSYILSTYATIKQEWEQWTMNNWIQELSKNSW (r-W371(ch. 8)) | SEQ ID NO 32 |
| WSNKSLEQIWNNMTWQEWEQKITAYTSLIHSLLEQAQ (W37Q(ch. 9)) | SEQ ID NO 33 |
| QAQELLSHILSTYATIKQEWEQWTMNNWIQELSKNSW (W371(ch. 9)) | SEQ ID NO 34 |
| WSNKSLEQIWNNMTWQEWEQKITALLEQAQ (W37Q(del. 7)) | SEQ ID NO 35 |
| QAQELLATIKQEWEQWTMNNWIQELSKNSW (r-W37Q(del. 7)) | SEQ ID NO 36 |
| FWNWLSAW (r-W8F) | SEQ ID NO 38 |
| ELDKWASLWNWFNITN (E16N) | SEQ ID NO 39 |
| NTINFWNWLSAWKDLE (r-E16N) | SEQ ID NO 40 |
| WNNMTWMEWEREIDNYTSL (W19L) | SEQ ID NO 41 |
| LSTYNDIEREWEMWTMNNW (r-W19L) | SEQ ID NO 42 |
| WNASWSNKSLEQIWNNMTWMEWEREIDNYTSLIYSLI (W37I) | SEQ ID NO 43 |
| ILSYILSTYNDIEREWEMWTMNNWIQELSKNSWSANW (r-W37I) | SEQ ID NO 44 |
| CSLEQIWNNMTWMEWEREIDNYTSLIYSLI (C30I) | SEQ ID NO 45 |
| ILSYILSTYNDIEREWEMWTMNNWIQELSC (r-C30I) | SEQ ID NO 46 |
| KSLEQIWNNMTWMEWEREIDNYTSLIYSLIK (K31K(A)) | SEQ ID NO 47 |
| KILSYILSTYNDIEREWEMWTMNNWIQELSK (r-K31K(A)) | SEQ ID NO 48 |
| KKLEQIWNNMTWMEWEREIDNYTSLIYSLKK (K31K(B)) | SEQ ID NO 49 |
| KKLSYILSTYNDIEREWEMWTMNNWIQELKK (r-K31K(B)) | SEQ ID NO 50 |
| WFNITNWLWY | SEQ ID NO 55 |
| YWLWNTINFW | SEQ ID NO 56 |
| bN31KAc-NMTWNFHLRPRDLISNINVIVLELKGSQQEK-NH2 | SEQ ID NO 57 |
| bS31E | |
| Ac-SLEQELKHLQCLEEELKPLEEVLNLAQLIEE-NH2 | SEQ ID NO 58 |
| bL31I | |
| Ac-LLQLKPMYFKFTLMRTLKPNKYNNIGNLLGI-NH2 | SEQ ID NO 59 |
| bI31L | |
| Ac-IGLLNGINNYKNPKLTRMLTFKFYMPKLQLL-NH2 | SEQ ID NO 60 |
| bQ31T | |
| Ac-QIWNNMTWTATIVEFLNRWITFCQSIISTLT-NH2 | SEQ ID NO 61 |
| bF31T | |
| Ac-FMCEYADETATIVEFLNRWITFCQSIISTLT-NH2 | SEQ ID NO 62 |
| bA31F | |
| Ac-AQSKNFHLRPRDLISNINVIVLELKGSETTF-NH2 | SEQ ID NO 63 |
| bK31F | |
| Ac-KKATELKHLQCLEEELKPLEEVLNLAQSKNF-NH2 | SEQ ID NO 64 |
| bE31Q | |
| Ac-ETAKKPMYFKFTLMRTLKPNKYNNIGNLTMQ-NH2 | SEQ ID NO 65 |
| bQ31E | |
| Ac-QMILNGINNYKNPKLTRMLTFKFYMPKKATE-NH2 | SEQ ID NO 66 |
| bW37Q(IL-2a2): | |
| Ac-WSNKSLEQELKHLQCLEEELKPLEEVLNLAQLIEESQ-NH2 | SEQ ID NO 67 |
| bW37(33)Q(IL-2a-g): | |
| Ac-WSNINNYKNPKLTRMLTFKFYMPLIYSLIEESQ-NH2 | SEQ ID NO 68 |
| bW37(34)Q(IL-2a-g): | |
| Ac-WSNDETATIVEFLNRWITFCQSIILIYSLIEESQ-NH2 | SEQ ID NO 69 |
| bN36(39)E(IL-2b1): | |
| Ac-NNMTWMEWEREIDNYTTQLQLEHLLLDLQMILNGINEQE-NH2 | SEQ ID NO 70 |
| bN36E(IL-2b2): | |
| Ac-NNMTWNFHLRPRDLISNINVIVLELKGSQQEKNEQE-NH2 | SEQ ID NO 71 |
| bN36E(IL-2g) | |
| Ac-NNMTWTATIVEFLNRWITFCQSIISTLTQQEKNEQE-NH2 | SEQ ID NO 72 |

### BRIEF DESCRIPTION OF THE FIGURES

FIGS. 1(A) and 1(B) show three dimensional representations of a portion of a Gp41 trimer and an IL-2 molecule with residues that are homologous in the two molecules highlghted.

FIG. 2. Regions of gp41 monomer showing synthetic HIV fusion inhibitory peptides according to the invention.

FIG. 3. Dose dilution inhibition of HIV-1 _{IIIB} infectivity by synthetic gp41-derived peptides S291, E30E (T-21 analogue) and Y36F (T-20 analogue).

FIG. 4. Dose dilution inhibition of HIV-1 infectivity in PBMCs by gp41-derived peptides S291, Y36F and control for primary isolates JRCSF, SF162 and patient isolate BR94 (clade B).

FIGS. 5(A) and 5(B) show activity of peptides according to the invention compared to T20 against HIV_{IIIB} and HIV_{BR} clades, respectively.

FIGS. 6(A)-(G) and (O) show IC50s for Peptides according to the invention in comparison to to T20, T1249 and AZT.

FIG. 7. Inhibition of cell-free virus infection.

FIG. 8(A-D). Inhibition curves for viral entry for HIV-1 chimeric envelope virus quantified by luciferase readout for gp41-derived synthetic peptides.

FIGS. 9(A and B). Effect of timing of peptide addition on inhibition of infection.

### DETAILED DESCRIPTION OF SELECTED EMBODIMENTS OF THE INVENTION

As discussed above, human immunodeficiency virus type 1 (HIV-1) envelope glycoprotein (Env) transmembrane subunit gp41 plays an important role in the early stage of HIV entry. The synthetic peptides designed according to the invention belong to a new class of antiretroviral compounds, which target a region in gp41 that is required for HIV-1 Env mediated fusion. A representative peptide of this new class, T-20, developed by Trimeris, Inc. and Hoffmann-La Roche, Ltd., has shown significant promise in the clinical trials and was approved by US FDA on Mar. 13, 2003 for treatment of patients infected by HIV-1, including the strains resistant to current anti-retrovirus drugs, i.e., reverse transcriptase and protease inhibitors.

Physical-chemical studies with synthetic peptides have indicated that the region targeted by T-20 prevents the formation of a six-helix bundle that is required for fusion. However, HIV-1 variants resistant to T-20 have emerged, which appear to have amino acid variations within the region targeted by T-20 and outside this region. The main challenge for the further development of this new class of antivirals is to overcome drug resistance.

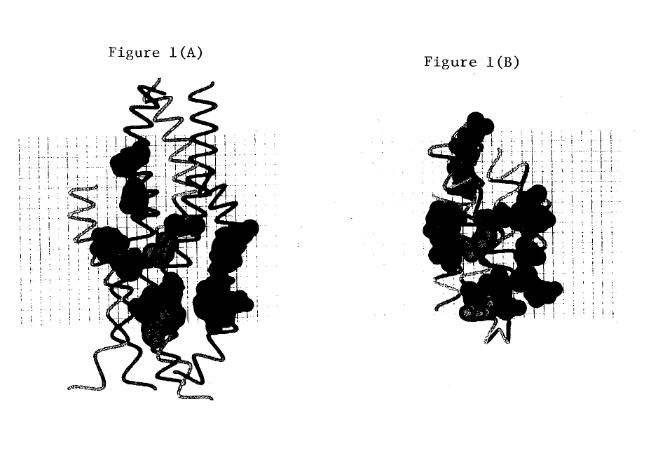

Accordingly, the present invention provides new peptides, which were rationally designed to enhance their activity in inhibiting HIV fusion to a target cell and minimize or completely eliminate drug resistance by making viral mutations difficult. The new peptides are based on a new approach that combines several aspects relating to sequence conservation among viral strains, structural features including three-dimensional similarity to regions of a host molecule such as IL-2 (see for example FIG. 1), helical content, distribution of hydrophobic and hydrophilic amino acids along the peptide, solvent accessibility and degree of theoretical immunogenicity.

The approach employed in designing the peptides of the invention yielded several dozens of new peptides, many of which have shown potency in vitro as inhibitors of HIV fusion to target cells. The analysis of the structure and activity of the designed peptides has allowed the unexpected appreciation by the inventors that a genus of peptides containing the sequence WXXXXWXWX, where W is a tryptophan and X is any amino acid have enhanced viral fusion inhibitory activity.

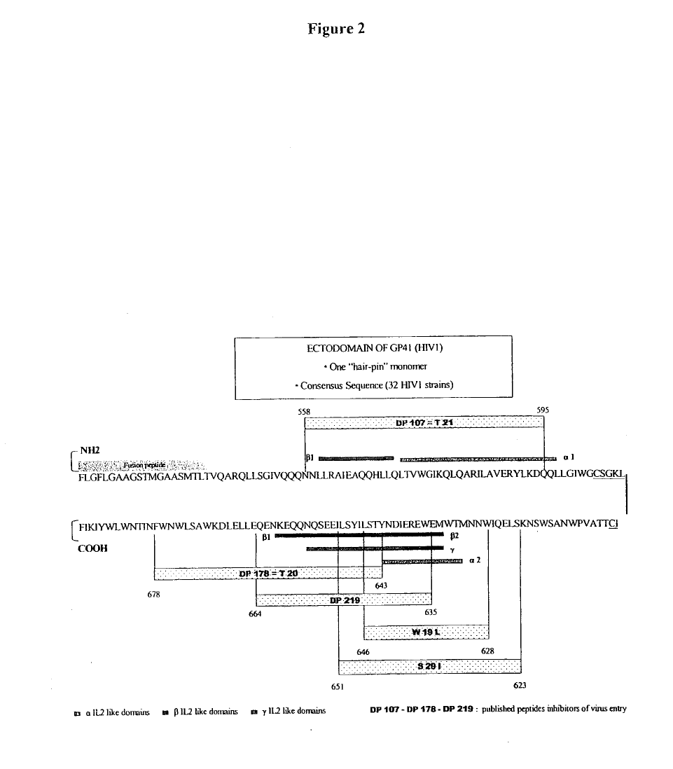

As well, the inventors have designed peptides based on a consensus sequence of the ectodomainm of Gp41 (FIG. 2) and its reverse sequence. The consensus sequences constructed by the inventors are as follows:

| | |
|---|---|
| VQARQLLSGIVQQQNNLLRAIEAQQHLLQLTVWGIKQ | (SEQ ID NO 1) |
| LQARILAVERYLKDQQLLGIWGCSGKLICTTAVPWNA | |
| SWSNKSLEQIWNNMTWMEWEREIDNYTSLIYSLIEES | |
| QNQQEKNEQELLELDKWASLWNWF | |
| FLGFLGAAGSTMGAASMTLTVQARQLLSGIVQQQNNL | (SEQ ID NO 2) |
| LRAIEAQQHLLQLTVWGIKQLQARILAVERYLKDQQL | |
| LGIWGCSGKLICTTAVPWNASWSNKSLEQIWNNMTWM | |
| EWEREIDNYTSLIYSLIEESQNQQEKNEQELLELDKW | |
| ASLWNWFNITNWLWYIKIF | |

Peptides designed according to the present invention have the following sequences:

| | |
|---|---|
| S29I sequences | |
| SLEQIWNNMTWMEWEREIDNYTSLIYSLI (S29I) | SEQ ID NO 3 |
| ILSYILSTYNDIEREWEMWTMNNWIQELS (r-S29I) | SEQ ID NO 4 |
| SLEQIWNNMT Nal2 MEWEREIDNYTSLIYSLI S29I(A) | SEQ ID NO 5 |
| ILSYILSTYNDIEREWEMNal2TMNNWIQELS (r-S29I(A)) | SEQ ID NO 6 |
| Ac-SLEQIWNNMTWME Nal2 EREIDNYTSLIYSLI (S29I(B)) | SEQ ID NO 7 |
| ILSYILSTYNDIEREWEMWT Nle NNWIQELS (r-S29I(B)) | SEQ ID NO 8 |
| SLEQIWNN Nle TWMEWEREIDNYTSLIYSLI (S29I(C)) | SEQ ID NO 9 |
| ILSYILSTYNDIEREWEMWT Nle NNWIQELS (r-S29I(C)) | SEQ ID NO 10 |
| Ac-SLEQI Nal2 NNMTWMEWEREIDNYTSLIYSLI (S29I(D)) | SEQ ID NO 11 |
| ILSYILSTYNDIEREWEMWTMNN Nal2 IQELS (r-S29I(D)) | SEQ ID NO 12 |
| SLEQIWNNMTW Nle EWEREIDNYTSLIYSLI (S29I(E)) | SEQ ID NO 13 |
| ILSYILSTYNDIEREWEMWTMNNWIQELS (r-S29I(E)) | SEQ ID NO 14 |
| M substituted by Nle = norleucine | |
| W substituted by Nal2 = napthyl-alanine | |
| (A = W11); (B = W14); (C = M9); (D = W6); (E = M12) | |
| N36E sequences | |
| NNMTWMEWEREIDNYTSLIYSLIEESQNQQEKNEQE (N36E) | SEQ ID NO 15 |
| EQENKEQQNQSEEILSYILSTYNDIEREWEMWTMNN (r-N36E) | SEQ ID NO 16 |
| NNMTWQEWEQKITAYTSLIYSLLEQAQIQQEKNEYE (N36E(ch. 10)) | SEQ ID NO 17 |
| EYENKEQQIQAQELLSYILSTYATIKQEWEQWTMNN (r-N36E(CH. 10)) | SEQ ID NO 18 |
| NNMTWQEWEQKITAYTSLIHSLLEQAQIQQEKNEYE (N36E(ch. 11)) | SEQ ID NO 19 |
| EYENKEQQIQAQELLSHILSTYATIKQEWEQWTMNN (r-N36E(CH. 11)) | SEQ ID NO 20 |
| NNMTWMEWEREIDNYTSLIHSLIEESQNQQEKNEQE (N36E(ch. 1)) | SEQ ID NO 21 |
| EQENKEQQNQSEEILSHILSTYNDIEREWEMWTMNN (r-N36E(ch. 1)) | SEQ ID NO 22 |
| NNMTWQEWEQKITALLEQAQIQQEKNEYE (N36E(del. 7)) | SEQ ID NO 23 |
| EYENKEQQIQAQELLATIKQEWEQWTMNN (r-N36E(del. 7)) | SEQ ID NO 24 |
| NNMTWQEWEQKITAYTSLIHSLIEESQNQQEKNEQE (N36E(ch. 6)) | SEQ ID NO 25 |
| EQENKEQQNQSEEILSHILSTYATIKQEWEQWTMNN (r-N36E(ch. 6)) | SEQ ID NO 26 |
| W37Q sequences | |
| WSNKSLEQIWNNMTWMEWEREIDNYTSLIYSLIEESQ (W37Q) | SEQ ID NO 27 |
| QSEEILSYILSTYNDIEREWEMWTMNNWIQELSKNSW (r-W37Q) | SEQ ID NO 28 |
| WSNKSLEQIWNNMTWMEWEREIDNYTSLIHSLIEESQ (W37Q(ch. 1)) | SEQ ID NO 29 |
| QSEEILSHILSTYNDIEREWEMWTMNNWIQELSKNSW (r-W37Q(ch. 1)) | SEQ ID NO 30 |
| WSNKSLEQIWNNMTWQEWEQKITAYTSLIYSLLEQAQ (W37Q(ch. 8)) | SEQ ID NO 31 |
| QAQELLSYILSTYATIKQEWEQWTMNNWIQELSKNSW (r-W371(ch. 8)) | SEQ ID NO 32 |
| WSNKSLEQIWNNMTWQEWEQKITAYTSLIHSLLEQAQ (W37Q(ch. 9)) | SEQ ID NO 33 |
| QAQELLSHILSTYATIKQEWEQWTMNNWIQELSKNSW (W371(ch. 9)) | SEQ ID NO 34 |
| WSNKSLEQIWNNMTWQEWEQKITALLEQAQ (W37Q(del. 7)) | SEQ ID NO 35 |
| QAQELLATIKQEWEQWTMNNWIQELSKNSW (r-W37Q(del. 7)) | SEQ ID NO 36 |
| W8F sequences | |
| WASLWNWF (W8F) | SEQ ID NO 37 |
| FWNWLSAW (r-W8F) | SEQ ID NO 38 |
| E16N sequences | |
| ELDKWASLWNWFNITN (E16N) | SEQ ID NO 39 |
| NTINFWNWLSAWKDLE (r-E16N) | SEQ ID NO 40 |
| W19L sequences | |
| WNNMTWMEWEREIDNYTSL (W19L) | SEQ ID NO 41 |
| LSTYNDIEREWEMWTMNNW (r-W19L) | SEQ ID NO 42 |
| W37I sequences | |
| WNASWSNKSLEQIWNNMTWMEWEREIDNYTSLIYSLI (W37I) | SEQ ID NO 43 |
| ILSYILSTYNDIEREWEMWTMNNWIQELSKNSWSANW (r-W37I) | SEQ ID NO 44 |
| C30I sequences | |
| CSLEQIWNNMTWMEWEREIDNYTSLIYSLI (C30I) | SEQ ID NO 45 |
| ILSYILSTYNDIEREWEMWTMNNWIQELSC (r-C30I) | SEQ ID NO 46 |
| K31K sequences | |
| KSLEQIWNNMTWMEWEREIDNYTSLIYSLIK (K31K(A)) | SEQ ID NO 47 |
| KILSYILSTYNDIEREWEMWTMNNWIQELSK (r-K31K(A)) | SEQ ID NO 48 |
| KKLEQIWNNMTWMEWEREIDNYTSLIYSLKK (K31K(B)) | SEQ ID NO 49 |
| KKLSYILSTYNDIEREWEMWTMNNWIQELKK (r-K31K(B)) | SEQ ID NO 50 |
| KKNNLLRAIEAQQHLLQLTVWGIKQLQARILAVERYLKDQKK (K42K) | SEQ ID NO 51 |
| YTSLIYSLIEESQNQQEKNEQELLELDKWASLWNWF (Y36F) | SEQ ID NO 52 |
| Compare to T20: | |
| YTSLIHSLIEESQNQQEKNEQELLELDKWASLWNWF (T20) | SEQ ID NO 53 |
| EWEREIDNYTSLIYSLIEESQNQQEKNEQE (E30E) | SEQ ID NO 54 |
| W10Y sequences | |
| WFNITNWLWY | SEQ ID NO 55 |
| YWLWNTINFW | SEQ ID NO 56 |
| bN31KAc-NMTWNFHLRPRDLISNINVIVLELKGSQQEK-NH2 | SEQ ID NO 57 |
| bS31E | |
| Ac-SLEQELKHLQCLEEELKPLEEVLNLAQLIEE-NH2 | SEQ ID NO 58 |
| bL31I | |
| Ac-LLQLKPMYFKFTLMRTLKPNKYNNIGNLLGI-NH2 | SEQ ID NO 59 |
| bI31L | |
| Ac-IGLLNGINNYKNPKLTRMLTFKFYMPKLQLL-NH2 | SEQ ID NO 60 |
| bQ31T | |
| Ac-QIWNNMTWTATIVEFLNRWITFCQSIISTLT-NH2 | SEQ ID NO 61 |
| bF31T | |
| Ac-FMCEYADETATIVEFLNRWITFCQSIISTLT-NH2 | SEQ ID NO 62 |
| bA31F | |
| Ac-AQSKNFHLRPRDLISNINVIVLELKGSETTF-NH2 | SEQ ID NO 63 |
| bK31F | |
| Ac-KKATELKHLQCLEEELKPLEEVLNLAQSKNF-NH2 | SEQ ID NO 64 |
| bE31Q | |
| Ac-ETAKKPMYFKFTLMRTLKPNKYNNIGNLIMQ-NH2 | SEQ ID NO 65 |
| bQ31E | |
| Ac-QMILNGINNYKNPKLTRMLTFKFYMPKKATE-NH2 | SEQ ID NO 66 |
| IL-2 based sequences | |
| bW37Q(IL-2a2): | |
| Ac-WSNKSLEQELKHLQCLEEELKPLEEVLNLAQLIEESQ-NH2 | SEQ ID NO 67 |
| bW37(33)Q(IL-2a-g): | |
| Ac-WSNINNYKNPKLTRMLTFKFYMPLIYSLIEESQ-NH2 | SEQ ID NO 68 |
| bW37(34)Q(IL-2a-g): | |
| Ac-WSNDETATIVEFLNRWITFCQSIILIYSLIEESQ-NH2 | SEQ ID NO 69 |
| bN36(39)E(IL-2b1): | |
| Ac-NNMTWMEWEREIDNYTTQLQLEHLLLDLQMILNGINEQE-NH2 | SEQ ID NO 70 |
| bN36E(IL-2b2): | |
| Ac-NNMTWNFHLRPRDLISNINVIVLELKGSQQEKNEQE-NH2 | SEQ ID NO 71 |
| bN36E(IL-2g) | |
| Ac-NNMTWTATIVEFLNRWITFCQSIISTLTQQEKNEQE-NH2 | SEQ ID NO 72 |

A panel of peptides according to the invention were examined for anti HIV-1 activity based on viral infection and cell fusion assays. The peptides were evaluated against two HIV-1 stains IIIB and BR92/92/021. These two stains were selected in part based on their co-receptor utilization preferences; HIV-1 IIIB utilizes the CXCR4 co-receptor whereas HIV-1 BR92 utilizes CCR5. To test anti-HIV efficacy, fresh Human Peripheral Blood Mononuclear Cells (PBMCs) were obtained from single, normal hepatitis and HIV-1 negative donors. The cells were stimulated by a using CD3+method. Drug testing was performed in a 96 well format in triplicate. Each plate contained control wells (cells+virus), experimental wells (drug+cells+virus) and compound control wells (drug+cells, to monitor cytotoxicity). The read-out for viral infectivity after seven days of infection was based on reverse transcriptase activity of cell free supernatant samples. Drug toxicity was determined in the control well by a standard assay using the tetrazolium compound, which was converted in live cells into a colored formazan product that can readily be monitored on the microtiter plate.

Peripheral blood mononuclear cells (PBMC) are isolated from healthy, uninfected donor blood for use in various assays and to culture HIV. The PBMC are stimulated with the mitogen phytohemagglutinin-P(PHA-P), in the presence of human interleukin 2 (IL-2) for 24-72 hours before use to promote blast formation and replication of T-cells. PBMCs were stimulated via the CD3 antigen that is part of the T cell receptor. This procedure results in the specific stimulation and enrichment of T cells, in contrast to PHA-P treatment, which results in the stimulation of both T cells and B cells. The T cell preparation contains about 50% CD4+cells that are susceptible to HIV-1 infection. Therefore no further separation between CD4+ and CD8+cells is necessary. Whole blood anticoagulated with heparin (120-240 mL), or leukocyte concentrates (buffy coats) is usually obtained from a blood bank, stored at room temperature and processed within 30 hours of collection. One such preparation is sufficient to perform the necessary assays.

A second panel of anti-HIV-1 peptides according to the invention was evaluated for using the MAGI-CCR5 antiviral assay and a gene reporter cell fusion assay. MAGI-CCR5 is an HIV-1 indicator cell line derived from HeLa cells that express CD4 and the HIV-1 coreceptors CCR5 and CXCR4. These cells contain a gene reporter cassette of β-galactosidase that is expressed from an HIV-1 LTR. Expression of the reporter gene is dependent on the production of HIV-1 tat that occurs following HIV-1 entry. The read-out for viral infectivity after two days of incubation is measurement of β-galactosidase expression detectable by chemoluminescence. The assay structure in the 96 well format as that used in the PBMC assay. The cell fusion assay uses the same indicator cell line (MAGI-CCR5), which is incubated with a HeLa cell based cell line HL2/3 that expresses HIV-1_{IIIB} Env on its surface and contains Tat and Rev expression cassettes. When HL2/3 cells fuse with the MAGI-CCR5 cells, HIV-1 Tat is delivered into the indicator cells leading to activation of the β-galactosidase gene which is under the control of the HIV-1 LTR. The enzyme activity is read out in a similar fashion by chemoluminescence using the same assay structure as mentioned above. This assay is uniquely sensitive to entry inhibitors.

The MAGI-CCR5 assay was done on two different days with peptides 23A, 54A and 6W8F, with reverse transcriptase inhibitor AZT and the CCR5 inhibitor, TAK-779. The cell fusion assays were performed on a subset of peptides in the same assay format. Three peptides according to the invention showed similar IC50 values (about 30 nM) as peptide 23A (T20).

Detailed description of tests involving peptides designed according to the invention is provided n the Examples below.

### EXAMPLE I

Methods

Peptide Inhibition Studies

Infectivity studies were performed using the inhibition of HIV-1 infection of the gp41 derived peptides using receptor bearing cells for both HIV-1 T cell line adapted virus (HIV-1 IIIB) in MT2 cells and primary isolate virus (SF162, JRCSF and 89.6) in peripheral blood lymphocytes. Both involved the culture of the virus with the peptide or its scrambled form for 1 hr at 37° C., 5% CO₂ and then subsequent addition of target cells for 7 days. Inhibition was assessed using an enzyme-linked immunosorbent assay (ELISA) of viral core protein p24 antigen.

Inhibition of Cell-Cell Fusion

Inhibition of HIV-1 infected cell fusion with receptor bearing cells was conducted using a semi-quantitative method using MT2 cells fusing to chronically infected H9 cells with HIV-1_{IIIB}. Inhibition of cell fusion by the peptides was assessed by scoring

β-galactosidase Cell Fusion Assay

HeLa-CD4 LTR-LacZ cells were seeded overnight at 6×10⁵ cells/ml. Chronically infected H9/HIV-1_{IIIB} cells (2×05 cells/ml) were pre-incubated with serial dilutions of peptide for 1 hr at 37° C., 5% CO₂ and then the mixture subsequently added to the adherent HeLa-CD4 cells. Overnight incubation (12 hrs) at 37° C., 5% CO₂ was followed by washing with PBS, trypsinisation and lysis with non-ionic detergent NP40. Subsequent addition of soluble substrate chlorophenol red β-D-galactopyranoside (CPRG) was followed by determination of optical density at 550 nm.

Flow Cytometry

Disruption/Inhibition of binding of gp41 antibodies (2F5 and 4E10) by the gp41-derived peptides were investigated using flow cytometry. H9 cells were infected with HIV-1_{IIIB} virus at a multiplicity of infection of ˜0.2 and cultured for 8-10 days, at which time were expressing CD4/gp120+as determined by flow cytometry. The chronically infected cells (1×10⁶ cells/ml) were incubated for 2 h at 37° C. with or without serial dilutions of gp41 peptides (S291 and Y36F and scrambled forms) and the antibodies 2F5 (5 ug/ml), 4E10 (10 ug/ml) and b12 (0.2 ug/ml). After washing (PBS/2% BSA/azide-FWB) the cells were fixed in 1% fomaldehyde in FWB for 2 h at 4° C. and subsequently labelled with anti-human phycoerythrin fluoresceinated conjugate antibody (30 min at 4° C.), washed×2 with FWB and then assayed by flow cytometry.

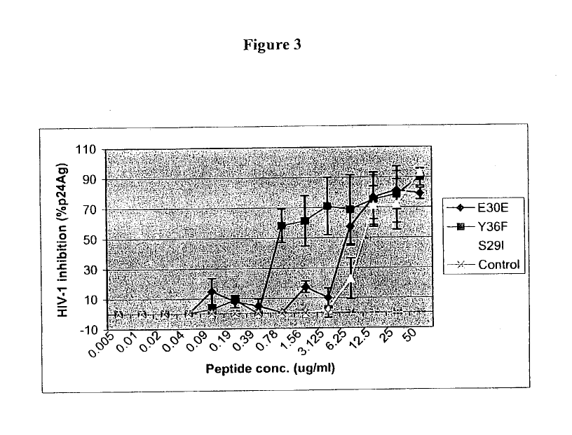

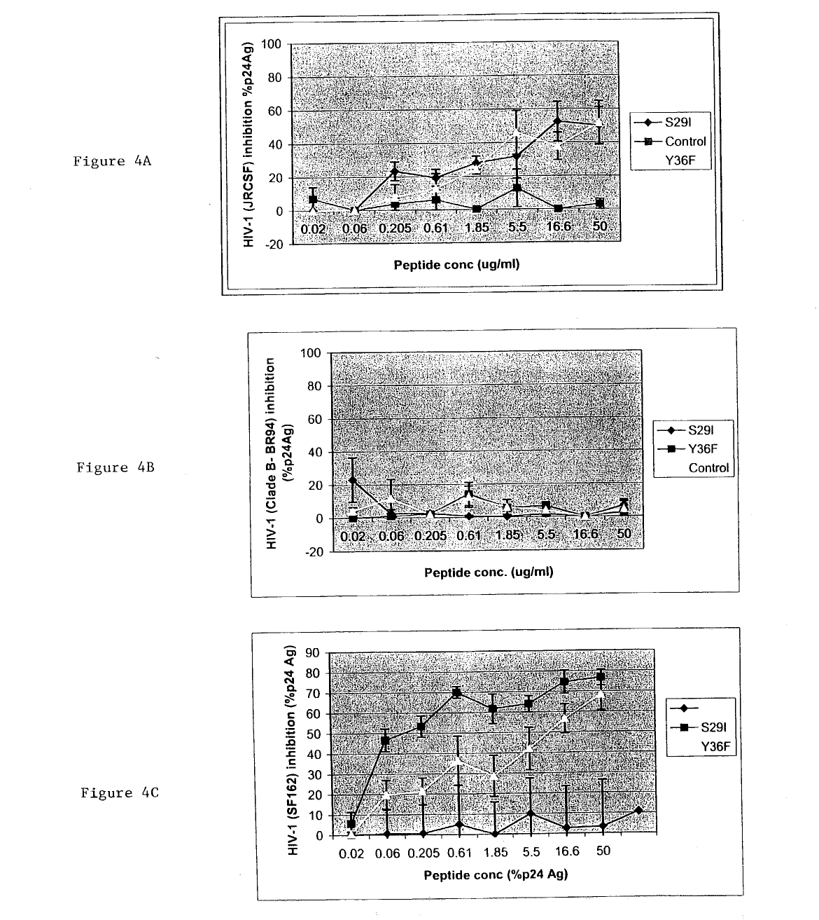

The results comparing the activity of the T20 and DP 219 analogues, Y36F and E30E to de novo designed peptide S291 are shown in Table 1 and FIGS. 3 and 4(A-C).

TABLE 1
ID50, ID90 and MIC values for inhibition of lab-adapted and primary
isolate HIV 1 infectivity for gp41 derived synthetic peptides.

| | HIV-1_{IIIB} infectivity | | | | | | | |
|---|---|---|---|---|---|---|---|---|
| | ID₅₀% | ID₉₀ | MIC | Primary isolate (ID₅₀) | | | | |
| Peptide | (μg/ml) | | | JRCSF | 89.6 | SF162 | BR94 | |
| Recombinant DP178 | 0.05 | 0.09 | 0.009 | | NT | | | |
| Synthesised DP178 | 4.69 | 24 | 0.80 | >50 | 50 | 30 | 0 | ✓ |
| Synthesised DP107 | — | — | — | | NT | | | — |
| Synthesised DP219 | 6.00 | 12.5 | 0.80 | | NT | | | ✓ |
| W19L | — | — | 0.06 | | NT | | | ? |
| S29I | 9.37 | 25 | 6.3 | >50 | 50 | 0.06 | 0 | ✓ |
| Control (I29I) | — | — | — | — | — | — | — | — |

### EXAMPLE II

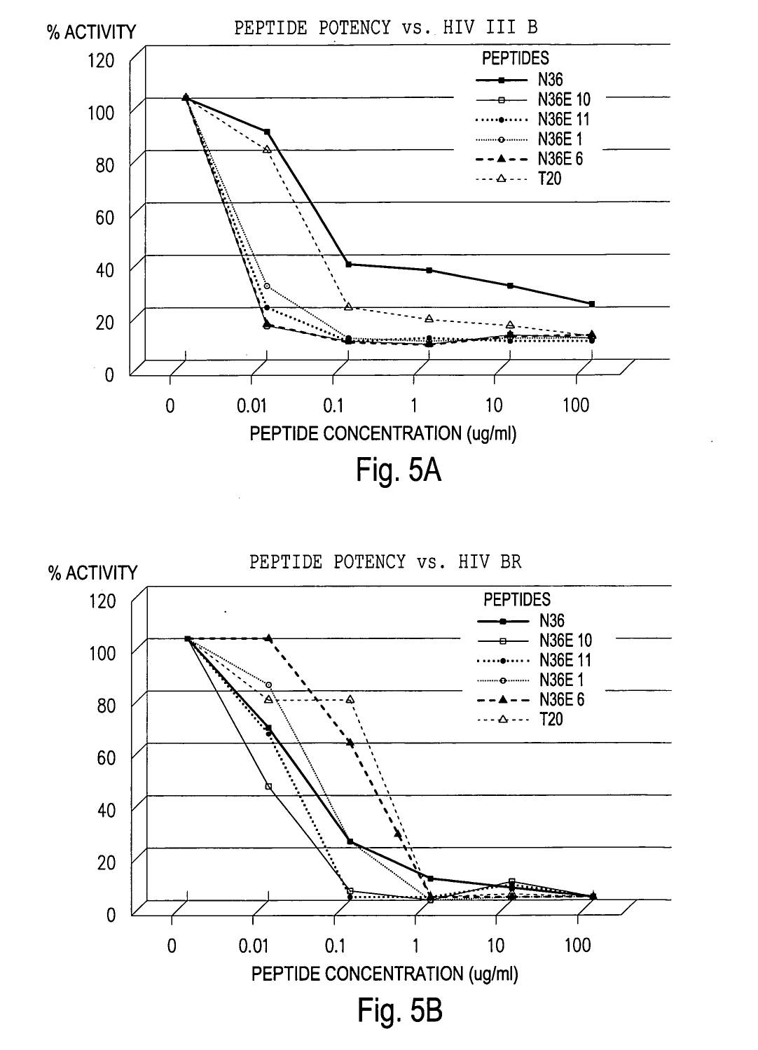

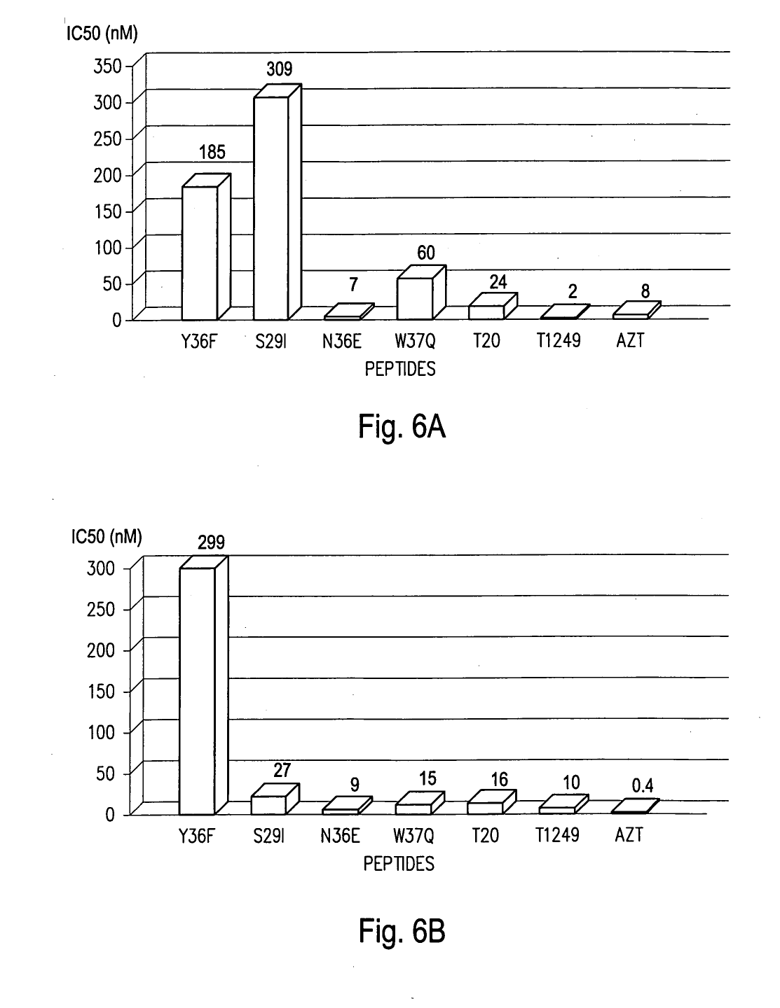

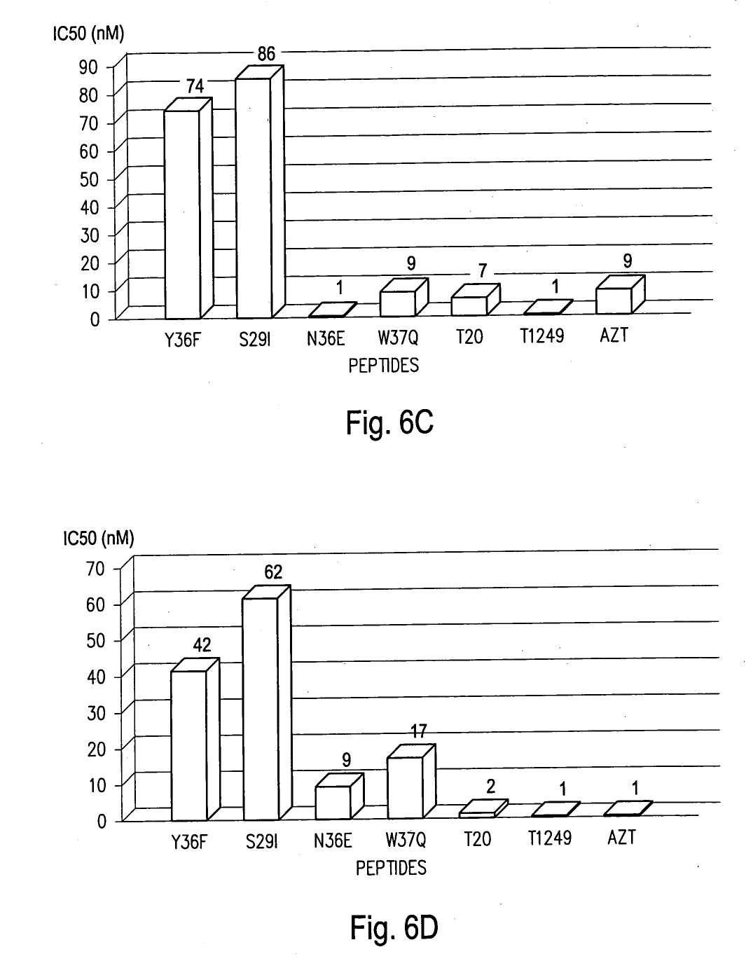

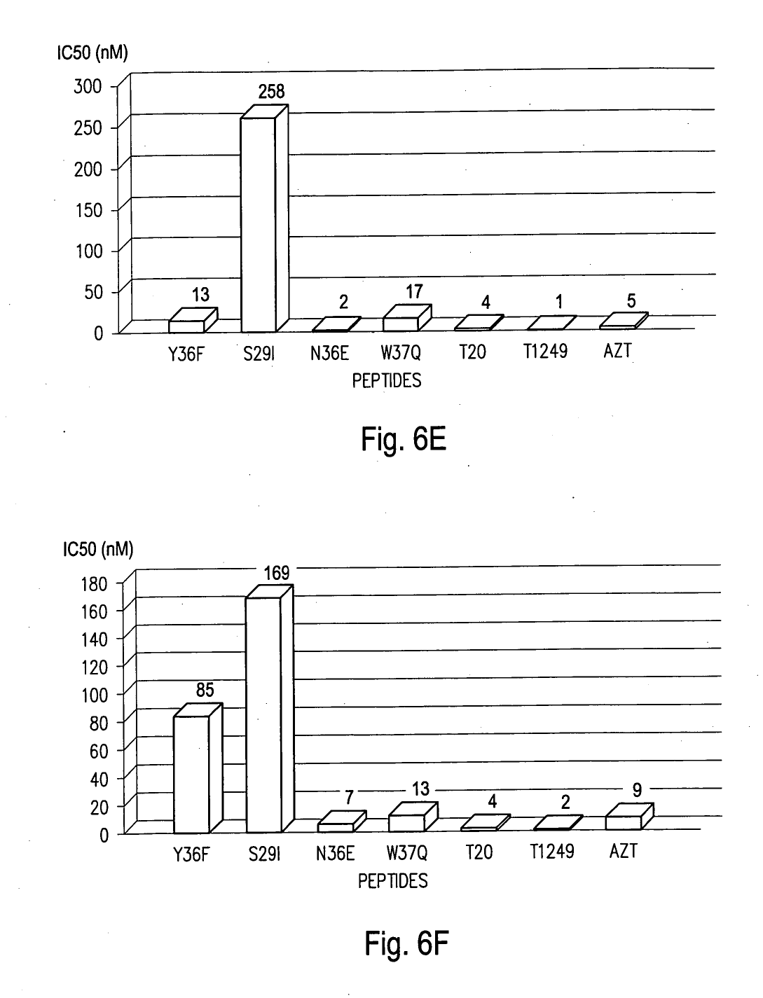

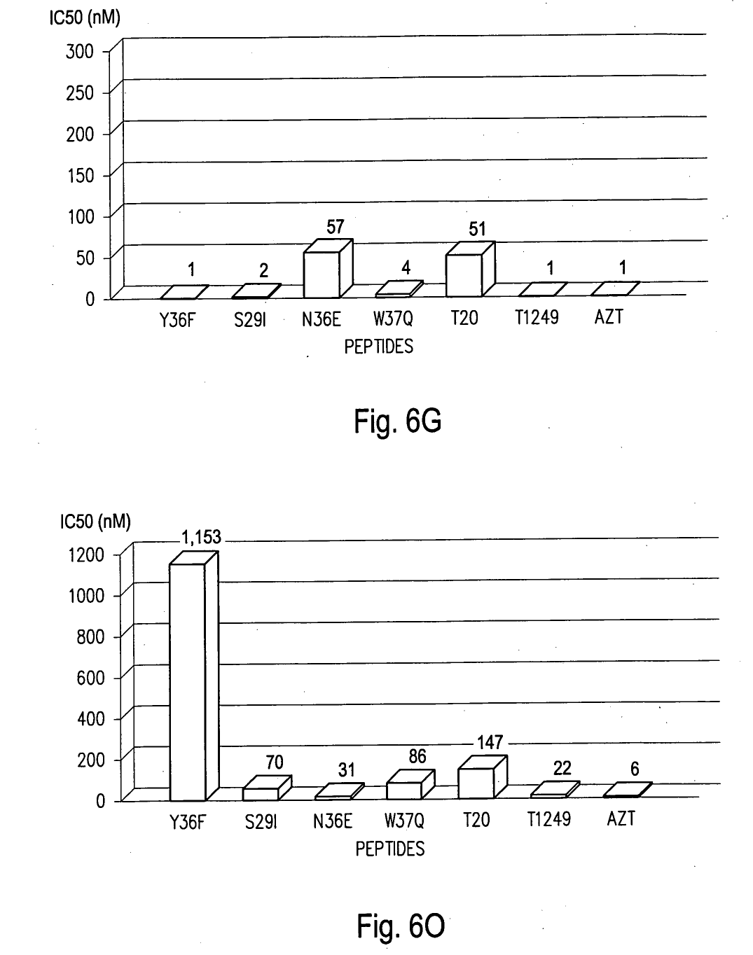

FIGS. 5(A), 5(B) and FIGS. 6(A)-(G) and 6(O) show comparisons of the activity across clades of de novo designed peptides according to the invention (N36E and variants thereof, S291 and W37Q), the analogue of T20 (Y36F), T20, T1249 and AZT. The data were obtained according to the following protocol:

Pre-incubate diluted peptides (0 to 50 ug/ml) with virus strain

Add 3 day anti-CD3 stimulated human peripheral blood mononuclear cells (50,000/well)

Incubate 37° C. for 6 days in IL-2 containing media

Test cell-free culture supernatants for reverse transcriptase (RT) activity at day 6

Test cells in cultures for toxicity using an MTS (CellTiter96 Reagent Promega) staining for cell viability

All assay results shown were done in triplicate

Peptides are derived from a consensus sequence of 32 strains of HIV-1

FIGS. 5(A), 5(B) and FIGS. 6(A)-(G) and 6(O), the present inventors have shown that peptides designed nationally based on the 3-D homology between Gp41 and IL-2 and other physical criteria can be very potent against various clades of the HIV virus. Particularly, the inventors have shown that:

An understanding of physical and structural homology between gp41 and IL-2 has allowed us to quickly and efficiently identify potent peptide molecules. Peptides derived from regions of high homology which are highly-conserved are potent.

The peptides disclosed herein showed activity against HIV-I isolates representative of the spectrum of known HIV variations from diverse geographic origins—Clades A, B, C, D, E, F, G, and 0.

Five peptides disclosed herein are active against HIV-IIIB & four active against BR/92/012 are more potent in vitro than T20.

Two peptides, N36E & W37Q, compare well to both T20 and to T1249

N36E in 6 independent assays in hPBMCs demonstrated an IC50 value of 2±1 nM

W37Q in 5 independent assays in hPBMCs demonstrated an IC50 value of 5±2 nM

Modifications to single amino acids, guided by our understanding of molecular “mimicry”, produce significant results in enhancing potency

### EXAMPLE III

In addition to the data discussed above in connection with Example I, De novo designed synthetic peptides according to the invention were further compared for anti fusion activity with novel analogues of T20, T21 and DP219. The new analogues are based on the consensus sequence described above and are therefore not identical to the previously known peptides and do fall within the scope of certain embodiments of the present invention. These analogues provide a benchmark for the impact of certain variations of the previously known peptides as well as an indication of how the other selected peptides according to the invention which were designed de novo would compare to previously reported peptides. It should be noted however, that the discussion provided below does not pertain to a direct comparison of peptides according to the invention with T20, T21 or DP219. Comparison to T20 and T1249 was provided above in Example II.

Peptides were tested in vitro for inhibition of early steps in cell-free HIV-1 infection and direct cell-to-cell spread of virus using T-cell line-adapted and primary isolates and recombinant HIV-1 viruses pseudotyped for env using CCR5, CXCR4 or both coreceptors. The peptides exhibited a spectrum of inhibitory activity in these assays and two of the de novo designed inhibitors (N36E and W37Q) were substantially more active than the T-20, T-21 and DP219 (new) analogues.

As discussed above, The human immunodeficiency virus type 1 (HIV-1) envelope glycoprotein (Env) is expressed in a trimeric form on the virion surface, with each monomer consisting of two noncovalently associated subunits: the gp120 surface glycoprotein and the gp41 transmembrane glycoprotein (Wyatt and Sodroski, 1998, Skehel and Wiley, 1998). The binding of gp120 to CD4 on the surface of a target cell induces a conformational change in gp120, which allows its subsequent interaction with a coreceptor, either CCR5 or CXCR4 (Berger et al, 1998). This secondary interaction is thought to result in a further conformational change in gp41 triggering the insertion of its hydrophobic amino-terminal fusion peptide into the target cell membrane, whilst the carboxy terminus remains anchored in the viral membrane. Gp41 contains an amino-terminal leucine/isoleucine heptad repeat (HR) region which has been shown crystallographically to form a triple-stranded α-helical central coiled-coil structure containing highly conserved grooves that the C-terminual peptides pack into in an antiparallel manner (Weissenhorn et al, 1997, Chan et al, 1997). A pre-fusion structure termed the pre-hairpin intermediate, whose molecular features are poorly understood, exists prior to the formation of the six-helix bundle. Peptide-based HIV-1 fusion inhibitors are thought to block HIV-1 fusion by interfering with formation of the six-helix bundle by binding to complementary target sequences along the NH₂-proximal heptad repeat (N—HR) or the COOH-proximal (C—HR) portion of gp41 (Jiang et al, 1993, Wild et al, 1995). This pre-hairpin intermediate target conformation is highly conserved and is present in the envelope proteins of several viruses such as influenza (Carr et al, 1993, Skehel and Wiley, 1998), simian immunodeficiency virus (Caffrey et al, 1998) and the Moloney murine leukaemia virus (Fass 1996). N—HR and C—HR-derived peptides are thought to block correct assembly of the six-helix bundle preventing apposition of the cellular and viral membranes therebye inhibiting fusion (Chan et al., 1998, Jiang et al, 1999, Rimsky et al, 1998, Kliger et al, 2001). N—HR peptides may also act by intercalating with the N—HR ectodomains of gp41 in a homotypic interaction

T20 (SEQ ID 53), also known as DP178 is a synthetic peptide corresponding to a 36-amino acid sequence of the C—HR region of gp41 that has potent HIV-1 inhibitory activity (Kliger 2000). It is reported to be effective at inhibiting HIV-1 fusion in vitro in a range of cellular types including dendritic cells and macrophages in addition to cultured cell lines and peripheral blood mononuclear cells (PBMC) (Ketas et al 2003). However, HIV-1 sensitivity to this peptide is somewhat modulated by coreceptor specificity, defined mainly by the V3 loop of gp120, and also a region within the gp41 (Derdeyn et al., 2000, 2001). It was shown in these studies that the mean 50% inhibitory concentration (IC₅₀) of T-20 for HIV-1 isolates using CCR5 for entry (R5 isolates) was 0.8 log¹⁰ higher than the mean IC₅₀ for CXCR4 isolates.

Selected de novo designed peptides according to the invention (S291, N36E, W37Q and W371) and the new analogues of T20, T21 and DP219 were evaluated for inhibition of both cell-free HIV-1 infection and of direct cell-to-cell spread using a range of T-cell line-adapted and primary isolates and recombinant HIV-1 pseudotyped for env derived from patient isolates of different clades. The peptides exhibited a range of activity in these assays and the de novo designed inhibitors were generally more active than the analogues of the previously described peptides. In particular these de novo designed peptides had comparable activity against both R5 and X4 HIV-1 isolates.

Inhibition of Cell-Free Virus Infection

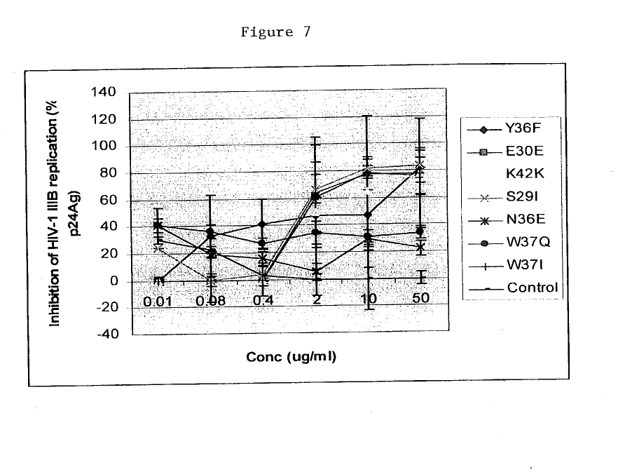

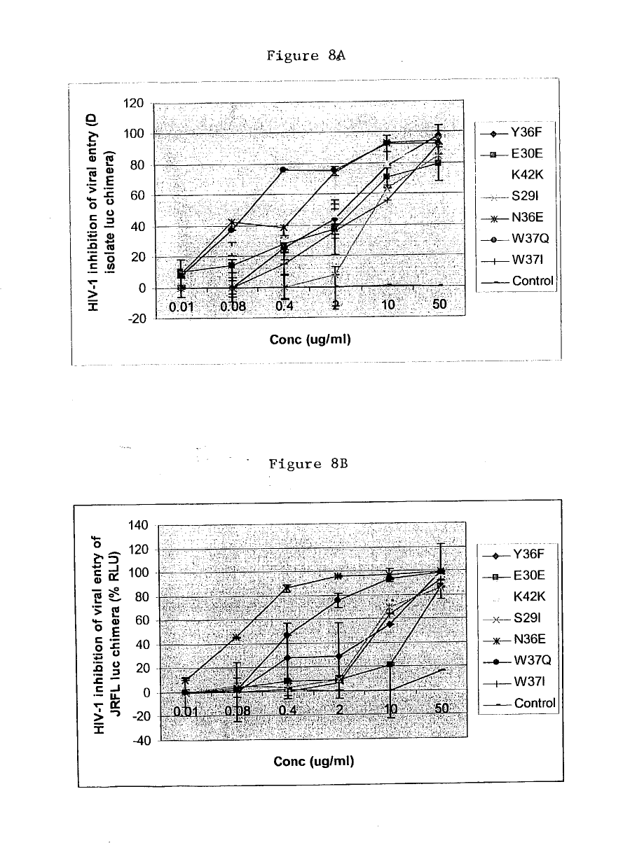

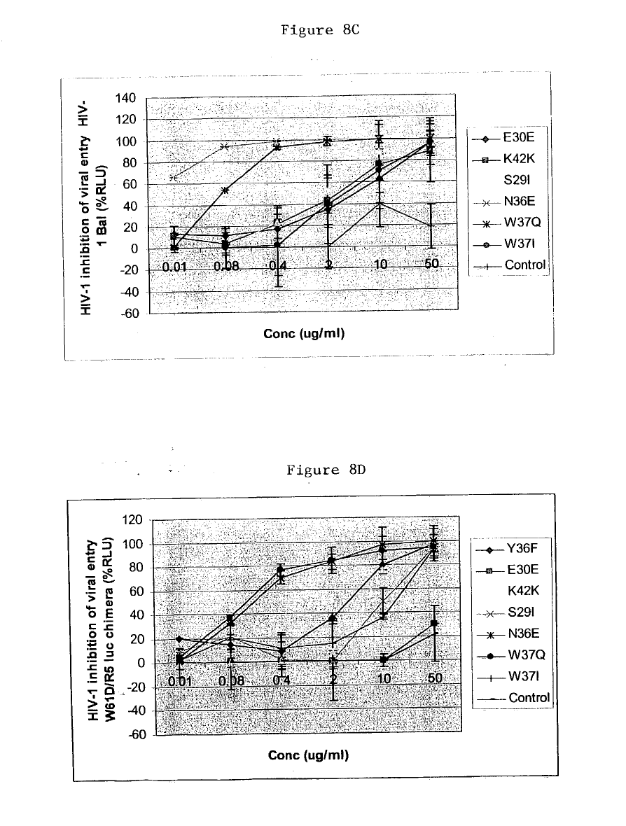

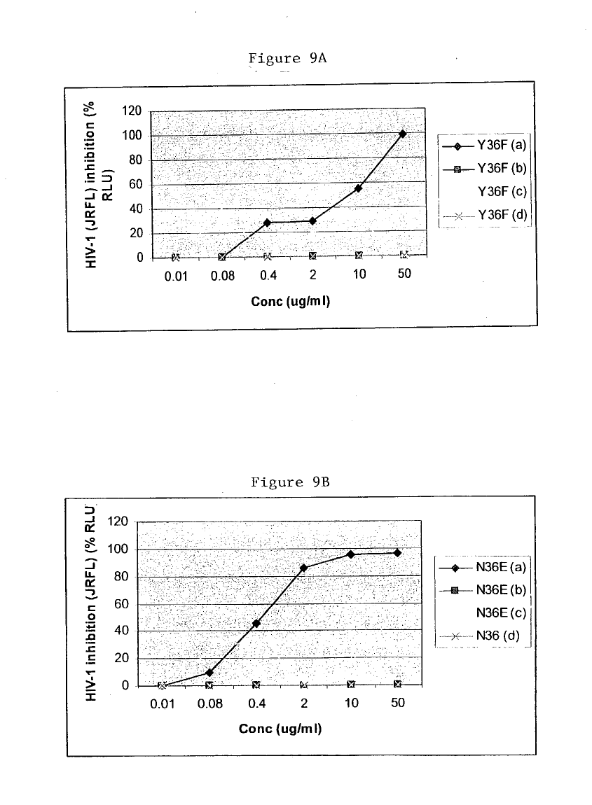

In preliminary experiments we tested the panel of peptides for inhibition of HIV-1 infectivity using a single CXCR4-using T cell line-adapted virus isolate: HIV-1_{IIIB}. Serial dilutions of the peptides were added to the target cells immediately followed by virus. After 5-7 days culture, supernatant was tested for viral p24 activity using a sensitive p24 ELISA. Results from a single representative experiment are shown in FIG. 7. All peptides showed some antiviral activity, the most potent being the three previously-described peptide analogues Y36F, K42K and E30E, and the de novo designed peptide W371. The 50% inhibitory concentrations (IC₅₀) for these peptides are shown in table 2: all are within a similar, mid-nanomolar range. Encouraged that all peptides were active against HIV-1, we moved on to test the panel against a series of recombinant pseudoviruses carrying Env from different tropic variants of HIV-1. These viruses carry a luciferase readout and are competent for a single cycle of replication only. We chose to test Env from four viruses representing: X4 TCLA virus (HXB2); X4 primary isolate (WHO D clade); R5X4 dual tropic primary isolate (W61D) and R5 primary isolates (JRFL and Bal). We observed significant dose-dependent inhibition of all viruses with all peptides with the exception of E30E on W61D (FIGS. 8A-D). The IC₅₀ data (table 3) indicate that W37Q was approximately 22-, 20- and 15-fold more active than Y36F, on the WHO D clade, W61D and JRFL pseudoviruses respectively. Similarly, N36E was 5-, 18- and 80-fold more active than Y36F (T-20 analogue) on HXB2, W61D (on CCR5+indicator cells) and JRFL pseudoviruses respectively. Moreover, both N36E and W37Q had IC₅₀ values of <0.1 on the Bal pseudovirus, indicating highly potent activity on a second R5 virus.

Cell-To-Cell Infection

HIV-1 transmission between infected and uninfected individuals and viral dissemination within an infected individual may take place by two distinct mechanisms: cell-free particles and direct cell-to-cell spread. We have devised an assay to quantify the inhibition of cell-to-cell spread of HIV-1 infection, based on the spread of virus directly from HIV-1 infected T cells to HeLa cells expressing CXCR4, CD4, CCR5, and a P-Gal-LTR readout (HeLa P5 cells). Co-incubation of HIV-1 infected T cells with HeLa P5 results in rapid transfer of virus to the HeLa P5 cells via cell-cell contact leading to activation of β-Gal, allowing a quantitative ELISA readout following addition of a soluble substrate to cell lysates. As with the cell-free virus inhibition assays, all peptides, with the exception of K42K on IIIB inhibited all three viruses. As before, N36E and W37Q were more potent than the T-20 analogue Y36F in terms of inhibition of cell-cell spread of Bal (R5) and 89.6 (R5×4) viruses. The other two de novo designed peptides, S291 and W371, inhibited in the mid-nanomolar range as before.

Timing of Peptide Addition and Inhibition of Infection

In order to gain some preliminary insight into the impact the time of addition of peptide with respect to the addition of virus to the target cells has on the activity of the peptides according to the invention, including the new analogues, we compared one of our more potent de novo designed peptides, N36E, with the T-20 analogue Y36F. Simultaneous mixing of the virus, cells and peptide as carried out in the previous experiments had the most pronounced inhibitory effect. Preincubation of the virus with the cells for 1 h prior to addition of the peptides resulted in complete absence of inhibition of pseudovirus infection, implying that the fusion process had proceeded past the point at which the peptides could interact with gp41. Preincubation of the virus with the peptides for 1 h at 37° C. followed by washing of the virus by ultrafiltration prior to addition to cells demonstrated some weak but poorly reproducible inhibition, suggesting that the peptides cannot react efficiently with virus in the absence of target cells. Finally, peptides preincubated with the target cells then washed prior to addition of virus had no inhibitory effect, confirming that the target structures of the peptides were probably not cellular.

The data presented herein suggest that these inhibitors are effective at reducing dissemination of HIV-1 in vivo both by cell-free virions and also in dense lymphoid tissue where cell-to-cell transfer may be the dominant mode of viral spread. We have compared our de novo designed peptides with analogues of three previously described peptides. The sequences of our peptide analogues of T-20, T-21 and DP219 are derived from the clade B consensus sequence (REF) and are therefore not identical to the originally described peptides that were derived from the HIV-1_{IIIB} sequence. Despite this, these peptide analogues had good antiviral activity similar to that described the original peptides. Comparison of the new analogues with de novo designed peptides demonstrated that S29I and W37I had broadly similar levels of activity, whereas N36E and W37Q displayed significantly greater potency. In some experiments N36E and W37Q were up to 100-fold more active than thew analogues Y36F, K42K and E30E, and were frequently 10-fold more potent.

Our time of addition studies with N36E, strongly suggest that this peptide acts at a stage of virus infection subsequent to virus-receptor binding but prior to entry. However, it appears that the window of opportunity for inhibition is short, since preincubation of virus and cells for 1 hour prior to addition of peptide was sufficient to abolish activity. These data are consistent with previous studies that propose peptide interaction with a transient ‘pre-haripin’ intermediate conformation that is present during gp120-CD4-coreceptor interaction but prior to formation of the six-helix bundle. It appears from these data that our de novo designed peptide N36E may interfere with HIV-1 fusion by a mechanism analogous or identical to that proposed for other C—HR peptides.

The potency of N36E and W37Q in vitro and their apparently similar activity on R5 and X4 viruses suggest that these peptides have promise as anti-HIV-1 therapeutic agents.

TABLE 2
Composite table for fifty percent inhibitory concentrations
(IC₅₀) for each peptide for HIV-1 viral
replication and cellular transfer of HIV-1

| | p24Ag | p24Ag | p24Ag | B-gal | B-gal | B-gal |
|---|---|---|---|---|---|---|
| PEPTIDE | IIIB | Bal | 89.6 | IIIB | Bal | 89.6 |
| Y36F | 2.0 | 9.0 | 7.5 | 1.9 | 0.8 | 5.01 |
| (T20) | | | | | | |
| K42K (T21) | 2.0 | NI | NI | NI | 9.01 | 0.35 |
| E30E | 1.6 | NI | NI | 1.45 | 18 | 5.00 |
| (DP219) | | | | | | |
| S29I | 1.45 | NI | 48 | 9 | 0.45 | 3.50 |
| N36E | — | NI | NI | | 0.32 | 0.24 |
| W37Q | — | NI | NI | | 0.065 | 1.15 |
| W37I | 1.50 | NI | NI | | 0.45 | 0.35 |

NI = no inhibition by peptide
ND = not performed

TABLE 3
Inhibition of viral entry

| | | Luc | | | Luc |
|---|---|---|---|---|---|
| | Luc | W61D | Luc | Luc | WHO isolate |
| | JRFL | (R5/ | HXB2 | Bal | Clade D |
| PEPTIDE | (R5) | X4) | (X4) | (R5) | (X4) |
| Y36F | 8.0 | 4.5/3 | 4 | ND | 2 |
| (T20) | | | | | |
| K42K (T21) | 7.1 | 6.8/2 | 2.5 | 3.5 | 5 |
| E30E | 28.3 | NI/NI | 5.0 | 6.0 | 2.5 |
| (DP219) | | | | | |
| S29I | 7.5 | 10.05/25 | 7.5 | 2.00 | 8 |
| N36E | 0.1 | 0.24/10 | 0.8 | <0.01 | 1.5 |
| W37Q | 0.55 | 0.21/3 | 0.18 | 0.07 | 0.5 |
| W37I | 8.0 | 20/5 | 7.0 | 4.5 | 0.8 |

What is claimed is:

1. A polypeptide having a sequence selected from the group consisting of:

| | |
|---|---|
| SLEQIWNNMTWMEWEREIDNYTSLIYSLI (S29I) | SEQ ID NO 3 |
| ILSYILSTYNDIEREWEMWTMNNWIQELS (r-S29I) | SEQ ID NO 4 |
| SLEQIWNNMT Nal2 MEWEREIDNYTSLIYSLI S29I(A) | SEQ ID NO 5 |
| ILSYILSTYNDIEREWEMNal2TMNNWIQELS (r-S29I(A)) | SEQ ID NO 6 |
| Ac-SLEQIWNNMTWME Nal2 EREIDNYTSLIYSLI (S29I(B)) | SEQ ID NO 7 |
| ILSYILSTYNDIEREWEMWT Nle NNWIQELS (r-S29I(B)) | SEQ ID NO 8 |
| SLEQIWNN Nle TWMEWEREIDNYTSLIYSLI (S29I(C)) | SEQ ID NO 9 |
| ILSYILSTYNDIEREWEMWT Nle NNWIQELS (r-S29I(C)) | SEQ ID NO 10 |
| Ac-SLEQI Nal2 NNMTWMEWEREIDNYTSLIYSLI (S29I(D)) | SEQ ID NO 11 |
| ILSYILSTYNDIEREWEMWTMNN Nal2 IQELS (r-S29I(D)) | SEQ ID NO 12 |
| SLEQIWNNMTW Nle EWEREIDNYTSLIYSLI (S29I(E)) | SEQ ID NO 13 |
| ILSYILSTYNDIEREWEMWTMNNWIQELS (r-S29I(E)) | SEQ ID NO 14 |
| NNMTWMEWEREIDNYTSLIYSLIEESQNQQEKNEQE (N36E) | SEQ ID NO 15 |
| EQENKEQQNQSEEILSYILSTYNDIEREWEMWTMNN (r-N36E) | SEQ ID NO 16 |
| NNMTWQEWEQKITAYTSLIYSLLEQAQIQQEKNEYE (N36E(ch. 10)) | SEQ ID NO 17 |
| EYENKEQQIQAQELLSYILSTYATIKQEWEQWTMNN (r-N36E(CH. 10)) | SEQ ID NO 18 |
| NNMTWQEWEQKITAYTSLIHSLLEQAQIQQEKNEYE (N36E(ch. 11)) | SEQ ID NO 19 |
| EYENKEQQIQAQELLSHILSTYATIKQEWEQWTMNN (r-N36E(CH. 11)) | SEQ ID NO 20 |
| NNMTWMEWEREIDNYTSLIHSLIEESQNQQEKNEQE (N36E(ch. 1)) | SEQ ID NO 21 |
| EQENKEQQNQSEEILSHILSTYNDIEREWEMWTMNN (r-N36E(ch. 1)) | SEQ ID NO 22 |
| NNMTWQEWEQKITALLEQAQIQQEKNEYE (N36E(del. 7)) | SEQ ID NO 23 |
| EYENKEQQIQAQELLATIKQEWEQWTMNN (r-N36E(del. 7)) | SEQ ID NO 24 |
| NNMTWQEWEQKITAYTSLIHSLIEESQNQQEKNEQE (N36E(ch. 6)) | SEQ ID NO 25 |
| EQENKEQQNQSEEILSHILSTYATIKQEWEQWTMNN (r-N36E(ch. 6)) | SEQ ID NO 26 |
| WSNKSLEQIWNNMTWMEWEREIDNYTSLIYSLIEESQ (W37Q) | SEQ ID NO 27 |
| QSEEILSYILSTYNDIEREWEMWTMNNWIQELSKNSW (r-W37Q) | SEQ ID NO 28 |
| WSNKSLEQIWNNMTWMEWEREIDNYTSLIHSLIEESQ (W37Q(ch. 1)) | SEQ ID NO 29 |
| QSEEILSHILSTYNDIEREWEMWTMNNWIQELSKNSW (r-W37Q(ch. 1)) | SEQ ID NO 30 |
| WSNKSLEQIWNNMTWQEWEQKITAYTSLIYSLLEQAQ (W37Q(ch. 8)) | SEQ ID NO 31 |
| QAQELLSYILSTYATIKQEWEQWTMNNWIQELSKNSW (r-W371(ch. 8)) | SEQ ID NO 32 |
| WSNKSLEQIWNNMTWQEWEQKITAYTSLIHSLLEQAQ (W37Q(ch. 9)) | SEQ ID NO 33 |
| QAQELLSHILSTYATIKQEWEQWTMNNWIQELSKNSW (W371(ch. 9)) | SEQ ID NO 34 |
| WSNKSLEQIWNNMTWQEWEQKITALLEQAQ (W37Q(del. 7)) | SEQ ID NO 35 |
| QAQELLATIKQEWEQWTMNNWIQELSKNSW (r-W37Q(del. 7)) | SEQ ID NO 36 |
| FWNWLSAW (r-W8F) | SEQ ID NO 38 |
| ELDKWASLWNWFNITN (E16N) | SEQ ID NO 39 |
| NTINFWNWLSAWKDLE (r-E16N) | SEQ ID NO 40 |
| WNNMTWMEWEREIDNYTSL (W19L) | SEQ ID NO 41 |
| LSTYNDIEREWEMWTMNNW (r-W19L) | SEQ ID NO 42 |
| WNASWSNKSLEQIWNNMTWMEWEREIDNYTSLIYSLI (W37I) | SEQ ID NO 43 |
| ILSYILSTYNDIEREWEMWTMNNWIQELSKNSWSANW (r-W37I) | SEQ ID NO 44 |
| CSLEQIWNNMTWMEWEREIDNYTSLIYSLI (C30I) | SEQ ID NO 45 |
| ILSYILSTYNDIEREWEMWTMNNWIQELSC (r-C30I) | SEQ ID NO 46 |
| KSLEQIWNNMTWMEWEREIDNYTSLIYSLIK (K31K(A)) | SEQ ID NO 47 |
| KILSYILSTYNDIEREWEMWTMNNWIQELSK (r-K31K(A)) | SEQ ID NO 48 |
| KKLEQIWNNMTWMEWEREIDNYTSLIYSLKK (K31K(B)) | SEQ ID NO 49 |
| KKLSYILSTYNDIEREWEMWTMTNNWIQELKK (r-K31K(B)) | SEQ ID NO 50 |
| WFNITNWLWY | SEQ ID NO 55 |
| YWLWNTINFW | SEQ ID NO 56 |
| bN31KAc-NMTWNFHLRPRDLISNINVIVLELKGSQQEK-NH2 | SEQ ID NO 57 |
| bS31E | |
| Ac-SLEQELKHLQCLEEELKPLEEVLNLAQLIEE-NH2 | SEQ ID NO 58 |
| bL31I | |
| Ac-LLQLKPMYFKFTLMRTLKPNKYNNIGNLLGI-NH2 | SEQ ID NO 59 |
| bI31L | |
| Ac-IGLLNGINNYKNPKLTRMLTFKFYMPKLQLL-NH2 | SEQ ID NO 60 |
| bQ31T | |
| Ac-QIWNNMTWTATIVEFLNRWITFCQSIISTLT-NH2 | SEQ ID NO 61 |
| bF31T | |
| Ac-FMCEYADETATIVEFLNRWITFCQSIISTLT-NH2 | SEQ ID NO 62 |
| bA31F | |
| Ac-AQSKNFHLRPRDLISNINVIVLELKGSETTF-NH2 | SEQ ID NO 63 |
| bK31F | |
| Ac-KKATELKHLQCLEEELKPLEEVLNLAQSKNF-NH2 | SEQ ID NO 64 |
| bE31Q | |
| Ac-ETAKKPMYFKFTLMRTLKPNKYNNIGNLIMQ-NH2 | SEQ ID NO 65 |
| bQ31E | |
| Ac-QMILNGINNYKNPKLTRMLTFKFYMPKKATE-NH2 | SEQ ID NO 66 |
| bW37Q(IL-2a2): | |
| Ac-WSNKSLEQELKHLQCLEEELKPLEEVLNLAQLIEESQ-NH2 | SEQ ID NO 67 |
| bW37(33)Q(IL-2a-g): | |
| Ac-WSNINNYKNPKLTRMLTFKFYMPLIYSLIEESQ-NH2 | SEQ ID NO 68 |
| bW37(34)Q(IL-2a-g): | |
| Ac-WSNDETATIVEFLNRWITFCQSIILIYSLIEESQ-NH2 | SEQ ID NO 69 |
| bN36(39)E(IL-2b1): | |
| Ac-NNMTWMEWEREIDNYTTQLQLEHLLLDLQMILNGINEQE-NH2 | SEQ ID NO 70 |
| bN36E(IL-2b2): | |
| Ac-NNMTWNFHLRPRDLISNINVIVLELKGSQQEKNEQE-NH2 | SEQ ID NO 71 |
| bN36E(IL-2g) | |
| Ac-NNMTWTATIVEFLNRWITFCQSIISTLTQQEKNEQE-NH2. | SEQ ID NO 72 |

2. A polypeptide having a sequence selected from the group consisting of:

| | |
|---|---|
| SLEQIWNNMTWMEWEREIDNYTSLIYSLI | SEQ ID NO 3 |
| (S29I) | |
| ILSYILSTYNDIEREWEMWTMNNWIQELS | SEQ ID NO 4 |
| (r-S29I) | |
| SLEQIWNNMT Nal2 MEWEREIDNYTSLIYSLI | SEQ ID NO 5 |
| S29I(A) | |
| ILSYILSTYNDIEREWEMNal2TMNNWIQELS | SEQ ID NO 6 |
| (r-S29I(A)) | |
| Ac-SLEQIWNNMTWME Nal2 EREIDNYTSLIYSLI | SEQ ID NO 7 |
| (S29I(B)) | |
| ILSYILSTYNDIEREWEMWT Nle NNWIQELS | SEQ ID NO 8 |
| (r-S29I(B)) | |
| SLEQIWNN Nle TWMEWEREIDNYTSLIYSLI | SEQ ID NO 9 |
| (S29I(C)) | |
| ILSYILSTYNDIEREWEMWT Nle NNWIQELS | SEQ ID NO 10 |
| (r-S29I(C)) | |
| Ac-SLEQI Nal2 NNMTWMEWEREIDNYTSLIYSLI | SEQ ID NO 11 |
| (S29I(D)) | |
| ILSYILSTYNDIEREWEMWTMNN Nal2 IQELS | SEQ ID NO 12 |
| (r-S29I(D)) | |
| SLEQIWNNMTW Nle EWEREIDNYTSLIYSLI | SEQ ID NO 13 |
| (S29I(E)) | |
| ILSYILSTYNDIEREWEMWTMNNWIQELS | SEQ ID NO 14 |
| (r-S29I(E)) | |

3. A polypeptide having a sequence selected from the group consisting of:

| | |
|---|---|
| NNMTWMEWEREIDNYTSLIYSLIEESQNQQEKNEQE | SEQ ID NO 15 |
| (N36E) | |
| EQENKEQQNQSEEILSYILSTYNDIEREWEMWTMNN | SEQ ID NO 16 |
| (r-N36E) | |
| NNMTWQEWEQKITAYTSLIYSLLEQAQIQQEKNEYE | SEQ ID NO 17 |
| (N36E(ch. 10)) | |
| EYENKEQQIQAQELLSYILSTYATIKQEWEQWTMNN | SEQ ID NO 18 |
| (r-N36E(CH. 10)) | |
| NNMTWQEWEQKITAYTSLIHSLLEQAQIQQEKNEYE | SEQ ID NO 19 |
| (N36E(ch. 11)) | |
| EYENKEQQIQAQELLSHILSTYATIKQEWEQWTMNN | SEQ ID NO 20 |
| (r-N36E(CH. 11)) | |
| NNMTWMEWEREIDNYTSLIHSLIEESQNQQEKNEQE | SEQ ID NO 21 |
| (N36E(ch. 1)) | |
| EQENKEQQNQSEEILSHILSTYNDIEREWEMWTMNN | SEQ ID NO 22 |
| (r-N36E(ch. 1)) | |
| NNMTWQEWEQKITALLEQAQIQQEKNEYE | SEQ ID NO 23 |
| (N36E(del. 7)) | |
| EYENKEQQIQAQELLATIKQEWEQWTMNN | SEQ ID NO 24 |
| (r-N36E(del. 7)) | |
| NNMTWQEWEQKITAYTSLIHSLIEESQNQQEKNEQE | SEQ ID NO 25 |
| (N36E(ch. 6)) | |
| EQENKEQQNQSEEILSHILSTYATIKQEWEQWTMNN | SEQ ID NO 26 |
| (r-N36E(ch. 6)) | |

4. A polypeptide having a sequence selected from the group consisting of:

| | | |
|---|---|---|
| bW37Q(IL-2a2): | Ac-WSNKSLEQELKHLQCLEEELKPLEEVLNLAQLIEESQ-NH2 | SEQ ID NO 67 |
| bW37(33)Q(IL-2a-g): | Ac-WSNINNYKNPKLTRMLTFKFYMPLIYSLIEESQ-NH2 | SEQ ID NO 68 |
| bW37(34)Q(IL-2a-g): | Ac-WSNDETATIVEFLNRWITFCQSIILIYSLIEESQ-NH2 | SEQ ID NO 69 |
| bN36(39)E(IL-2b1): | Ac-NNMTWMEWEREIDNYTTQLQLEHLLLDLQMILNGINEQE-NH2 | SEQ ID NO 70 |
| bN36E(IL-2b2): | Ac-NNMTWNFHLRPRDLISNINVIVLELKGSQQEKNEQE-NH2 | SEQ ID NO 71 |
| bN36E(IL-2g) | Ac-NNMTWTATIVEFLNRWITFCQSIISTLTQQEKNEQE-NH2 | SEQ ID NO 72 |

5. A polypeptide having a sequence selected from the group consisting of:

| | |
|---|---|
| WSNKSLEQIWNNMTWMEWEREIDNYTSLIYSLIEESQ | SEQ ID NO 27 |
| (W37Q) | |
| QSEEILSYILSTYNDIEREWEMWTMNNWIQELSKNSW | SEQ ID NO 28 |
| (r-W37Q) | |
| WSNKSLEQIWNNMTWMEWEREIDNYTSLIHSLIEESQ | SEQ ID NO 29 |
| (W37Q(ch. 1)) | |
| QSEEILSHILSTYNDIEREWEMWTMNNWIQELSKNSW | SEQ ID NO 30 |
| (r-W37Q(ch. 1)) | |
| WSNKSLEQIWNNMTWQEWEQKITAYTSLIYSLLEQAQ | SEQ ID NO 31 |
| (W37Q(ch. 8)) | |
| QAQELLSYILSTYATIKQEWEQWTMNNWIQELSKNSW | SEQ ID NO 32 |
| (r-W371(ch. 8)) | |
| WSNKSLEQIWNNMTWQEWEQKITAYTSLIHSLLEQAQ | SEQ ID NO 33 |
| (W37Q(ch. 9)) | |
| QAQELLSHILSTYATIKQEWEQWTMNNWIQELSKNSW | SEQ ID NO 34 |
| (W371(ch. 9)) | |
| WSNKSLEQIWNNMTWQEWEQKITALLEQAQ | SEQ ID NO 35 |
| (W37Q(del. 7)) | |
| QAQELLATIKQEWEQWTMNNWIQELSKNSW | SEQ ID NO 36 |
| (r-W37Q(del. 7)) | |

6. A polypeptide having a sequence selected from the group consisting of:

| bN31KAc-NMTWNFHLRPRDLISNINVIVLELKGSQQEK-NH2 | | SEQ ID NO 57 |
|---|---|---|
| bS31E | Ac-SLEQELKHLQCLEEELKPLEEVLNLAQLIEE-NH2 | SEQ ID NO 58 |
| bL31I | Ac-LLQLKPMYFKFTLMRTLKPNKYNNIGNLLGI-NH2 | SEQ ID NO 59 |
| bI31L | Ac-IGLLNGINNYKNPKLTRMLTFKFYMPKLQLL-NH2 | SEQ ID NO 60 |
| bQ31T | Ac-QIWNNMTWTATIVEFLNRWITFCQSIISTLT-NH2 | SEQ ID NO 61 |
| bF31T | Ac-FMCEYADETATIVEFLNRWITFCQSIISTLT-NH2 | SEQ ID NO 62 |
| bA31F | Ac-AQSKNFHLRPRDLISNINVIVLELKGSETTF-NH2 | SEQ ID NO 63 |
| bK31F | Ac-KKATELKHLQCLEEELKPLEEVLNLAQSKNF-NH2 | SEQ ID NO 64 |
| bE31Q | Ac-ETAKKPMYFKFTLMRTLKPNKYNNIGNLIMQ-NH2 | SEQ ID NO 65 |

7. A polypeptide having a sequence selected from the group consisting of:

| | |
|---|---|
| FWNWLSAW (r-W8F) | SEQ ID NO 38 |
| ELDKWASLWNWFNITN (E16N) | SEQ ID NO 39 |
| NTINFWNWLSAWKDLE (r-E16N) | SEQ ID NO 40 |
| WNNMTWMEWEREIDNYTSL (W19L) | SEQ ID NO 41 |
| LSTYNDIEREWEMWTMNNW (r-W19L) | SEQ ID NO 42 |
| WNASWSNKSLEQIWNNMTWMEWEREIDNYTSLIYSLI | SEQ ID NO 43 |
| (W37I) | |
| ILSYILSTYNDIEREWEMWTMNNWIQELSKNSWSANW | SEQ ID NO 44 |
| (r-W37I) | |
| CSLEQIWNNMTWMEWEREIDNYTSLIYSLI | SEQ ID NO 45 |
| (C30I) | |
| ILSYILSTYNDIEREWEMWTMNNWIQELSC | SEQ ID NO 46 |
| (r-C30I) | |
| KSLEQIWNNMTWMEWEREIDNYTSLIYSLIK | SEQ ID NO 47 |
| (K31K(A)) | |
| KILSYILSTYNDIEREWEMWTMNNWIQELSK | SEQ ID NO 48 |
| (r-K31K(A)) | |
| KKLEQIWNNMTWMEWEREIDNYTSLIYSLKK | SEQ ID NO 49 |
| (K31K(B)) | |
| KKLSYILSTYNDIEREWEMWTMNNWIQELKK | SEQ ID NO 50 |
| (r-K31K(B)) | |
| WFNITNWLWY | SEQ ID NO 55 |
| YWLWNTINFW | SEQ ID NO 56 |

8. A polypeptide comprising the sequence WXXXXWXWX, where X is any amino acid and W is tryptophan and the peptide inhibits HIV fusion to a target cell.

9. A method of inhibiting HIV fusion to a target cell comprising administering an inhibitory effective amount of a peptide of claim 1.

10. A method of inhibiting HIV fusion to a target cell comprising administering an inhibitory effective amount of a peptide of claim 8.

11. A method of treating a subject infected with the HIV virus, comprising administering to the subject a therapeutically effective amount of a peptide of claim 1.

12. A method of treating a subject infected with the HIV virus, comprising administering to the subject a therapeutically effective amount of a peptide of claim 8.
